(19) United States
(12) Patent Application Publication
(10) Pub. No.: US 2023/0073299 A1
(43) Pub. Date: Mar. 9, 2023

(54) SYSTEMS AND METHODS FOR MAPPING LOCAL ACTIVATION TIMES

(71) Applicant: St. Jude Medical, Cardiology Division, Inc., St. Paul, MN (US)

(72) Inventors: Valtino X. Afonso, Oakdale, MN (US); Nathaniel Bird, Minneapolis, MN (US); Patrick Kasi, Woodbury, MN (US); Jatin Relan, Bordeaux, Aquitaine (FR); Steven Kim, New York, NY (US)

(21) Appl. No.: 17/802,273

(22) PCT Filed: Dec. 9, 2020
(86) PCT No.: PCT/US2020/063871

§ 371 Date: Aug. 25, 2022

Related U.S. Application Data
(60) Provisional application No. 62/982,141, filed on Feb. 27, 2020.

(51) Int. Cl. A61B 5/367; A61B 5/287; A61B 5/339; G16H 40/63

(52) U.S. Cl. CPC A61B 5/367; A61B 5/287; A61B 5/339; G16H 40/63

(57) ABSTRACT

The present disclosure provides systems and methods for generating a local activation time (LAT) map. A method includes receiving at least one reference electrogram, receiving at least one roving electrogram, detecting cardiac activations in the at least one reference electrogram, detecting roving cardiac activations in the at least one roving electrogram, identifying, at a trigger time, a most recent reference cardiac activation of the detected reference cardiac activations, identifying, a corresponding roving cardiac activation of the detected roving cardiac activations that is closest in time to the most recent reference cardiac activation, the corresponding roving cardiac activation identified independent of any roving activation interval (RAI), computing, a LAT as a time difference between the most recent reference cardiac activation and the corresponding roving cardiac activation, and generating and displaying, a LAT map based on the computed LAT.

### CROSS REFERENCE TO RELATED APPLICATIONS

This application claims priority to provisional application Ser. No. 62/982,141, filed Feb. 27, 2020, which is incorporated herein by reference in its entirety.

### FIELD OF THE DISCLOSURE

The present disclosure relates generally to electrocardiography. In particular, in many embodiments, the present disclosure relates to systems and methods for mapping local activation times.

### BACKGROUND

It is generally known, in physiology, that cells undergo periodic depolarization and repolarization that is essential to the functioning of and communication among those cells. Electrocardiography is a technology by which cardiac electrical activity is monitored and recorded over time. Generally, the depolarization and repolarization patterns of the heart are detectable as small changes in charge in skin cells that are measured using, for example, various cutaneous electrodes. A graph of these charges, i.e., voltages, is referred to as an electrocardiogram (ECG). ECGs are often used to measure rate and rhythm of heartbeats, as well as to evaluate the cardiac cells to detect damage or diagnose potential heart conditions. Additionally, in electrophysiological procedures, an array of electrodes located on a distal end of a cardiac catheter is placed on the cardiac muscle to produce an electrogram.

Each electrode of the ECG and electrogram produces ECG and electrogram traces. A fundamental aspect of the ECG and electrogram is the accurate detection of cardiac activations in each trace. Such detections are an ongoing challenge in creating useful products from an ECG and electrogram.

For example, at least some known commercial cardiac mapping systems facilitate creating a map of the local activation time (LAT). Generally, to create this map, in at least some known commercial cardiac mapping systems, a user defines a roving catheter and a reference catheter. The user also defines a roving activation interval (RAI). The RAI is an interval of time relative to cardiac activity identified in the reference catheter. The cardiac mapping system, by design, uses the RAI to automatically reject cardiac activity falling outside of the RAI.

However, using the RAI to generate LAT maps may, in some scenarios, impair the accuracy of the generated maps. For example, in at least some known systems, cardiac cycle length (CCL) is measured and used to determine a time interval of the RAI. However, there are multiple, debated approaches for determining the time interval. In addition, user-defined RAIs may vary, for example, from 95% to 110% of the CCL (commonly 0.2 seconds to 0.5 seconds).

Further, user-defined RAIs are generally placed relative to cardiac activity sensed by the reference catheter. However, there are multiple debated approaches for placement of the RAI. For example, a user may place the RAI left of, centered on, or right of the sensed cardiac activity. Also, the CCL may be variable, even in cardiac rhythms considered to be regular. This can result in desired cardiac activity falling on a boundary of, or outside of the RAI, which may result in cardiac activity being missed by automatic detection algorithms constrained to process data within the RAI only.

LAT measurements used to generate the LAT map are based on a timing difference between a reference activation and a roving activation. Accordingly, in some scenarios, using a RAI results in improperly computing a LAT by using cardiac activity from two different cardiac cycles. Thus, given the disparate approaches and possible errors introduced when using a RAI, it would be desirable to generate LAT maps without using a RAI.

### BRIEF SUMMARY OF THE DISCLOSURE

In one embodiment, the present disclosure provides a method of generating a local activation time (LAT) map. The method includes receiving, at a computing system, at least one reference electrogram, receiving, at the computing system, at least one roving electrogram, detecting, using the computing system, reference cardiac activations in the at least one reference electrogram, detecting, using the computing system, roving cardiac activations in the at least one roving electrogram, identifying, using the computing system, at a trigger time, a most recent reference cardiac activation of the detected reference cardiac activations, identifying, using the computing system, a corresponding roving cardiac activation of the detected roving cardiac activations that is closest in time to the most recent reference cardiac activation, the corresponding roving cardiac activation identified independent of any roving activation interval (RAI), computing, using the computing system, a LAT as a time difference between the most recent reference cardiac activation and the corresponding roving cardiac activation, and generating and displaying, using the computing system, a LAT map based on the computed LAT.

In another embodiment, the present disclosure provides a computing system for use in generating a local activation time (LAT) map. The computing system includes a memory, and a processor communicatively coupled to the memory. The processor is configured to receive at least one reference electrogram, receive at least one roving electrogram, detect reference cardiac activations in the at least one reference electrogram, detect roving cardiac activations in the at least one roving electrogram, identify, at a trigger time, a most recent reference cardiac activation of the detected reference cardiac activations, identify a corresponding roving cardiac activation of the detected roving cardiac activations that is closest in time to the most recent reference cardiac activation, the corresponding roving cardiac activation identified independent of any roving activation interval (RAI), compute a LAT as a time difference between the most recent reference cardiac activation and the corresponding roving cardiac activation, and generate and display a LAT map based on the computed LAT.

In yet another embodiment, the present disclosure provides a cardiac mapping system for use in generating a local activation time (LAT) map. The system includes at least one reference catheter, at least one roving catheter, and a computing system communicatively coupled to the at least one reference catheter and the at least one roving catheter. The computing system is configured to receive at least one reference electrogram recorded by the at least one reference catheter, receive at least one roving electrogram recorded by the at least one roving catheter, detect reference cardiac activations in the at least one reference electrogram, detect roving cardiac activations in the at least one roving electrogram, identify, at a trigger time, a most recent reference cardiac activation of the detected reference cardiac activations, identify a corresponding roving cardiac activation of the detected roving cardiac activations that is closest in time to the most recent reference cardiac activation, the corresponding roving cardiac activation identified independent of any roving activation interval (RAI), compute a LAT as a time difference between the most recent reference cardiac activation and the corresponding roving cardiac activation, and generate and display a LAT map based on the computed LAT.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic diagram of a system for conducting an electrocardiogram;

FIG. 2 is a schematic diagram of the system of FIG. 1 having a catheter;

FIG. 3A is a schematic diagram of an exemplary catheter system for use in the system shown in FIG. 1 and FIG. 2;

FIG. 3B is a schematic diagram of an exemplary electrode assembly for use in the catheter system shown in FIG. 3A;

FIG. 4A is an illustration of an electrogram recorded by a reference catheter;

FIG. 4B is an illustration of an electrogram recorded by a roving catheter;

FIG. 4C is a schematic diagram illustrating acquiring sequential electrograms using a roving catheter;

FIG. 5 is a diagram illustrating computation of a local activation time (LAT) using a roving activation interval (RAI);

FIG. 6 is a diagram illustrating another example computation of a LAT using a RAI;

FIG. 7 is a flow diagram of an exemplary method of generating LAT maps;

FIG. 8 is a diagram illustrating computation of a LAT without using a RAI;

FIG. 9 is a diagram illustrating computing LAT sequentially without using a RAI;

FIG. 10 is a diagram illustrating identifying an invalid LAT without using a RAI;

FIG. 11 is a diagram illustrating a reference electrogram; and

FIG. 12 is a diagram illustrating ignoring certain reference cardiac activations when computing LAT and generating an LAT map.

Corresponding reference characters indicate corresponding parts throughout the several views of the drawings. It is understood that that Figures are not necessarily to scale.

### DETAILED DESCRIPTION OF THE DISCLOSURE

The systems and methods described herein enable generating a local activation time (LAT) map. A method includes receiving at least one reference electrogram, receiving at least one roving electrogram, detecting cardiac activations in the at least one reference electrogram, and detecting roving cardiac activations in the at least one roving electrogram. The method further includes identifying, at a trigger time, a most recent reference cardiac activation of the detected reference cardiac activations, and identifying, a corresponding roving cardiac activation of the detected roving cardiac activations that is closest in time to the most recent reference cardiac activation. The corresponding roving cardiac activation is identified independent of any roving activation interval (RAI). The method further includes computing a LAT as a time difference between the most recent reference cardiac activation and the corresponding roving cardiac activation, and generating and displaying, a LAT map based on the computed LAT.

The embodiments described herein enable automatically computing local activation times (LATs) and generating a LAT map without using a user-defined roving activation interval (RAI). Further, the systems and methods described herein mitigate disadvantages of at least some known cardiac mapping systems. For example, in at least some known cardiac mapping systems, a user defines a reference catheter from which reference cardiac activations are automatically determined, and the user sets a RAI, relative to the reference cardiac activations. However, the RAI may result in some desired cardiac activations being missed, as described herein. Further, in at least some known cardiac mapping systems, far-field cardiac activations may cause incorrect detection of a cardiac activation, resulting in incorrect LAT computations.

In contrast, the systems and methods described herein have a number of advantages. Although the user still defines a reference catheter from which reference cardiac activations are automatically determined, the user does not need to set a RAI in the embodiments described herein. Instead, the user sets or selects an observation time (e.g., in a range from 0.1 to 8.0 seconds), within which a roving catheter is held stationary to facilitate mapping cardiac anatomy. Cardiac activations are detected over the observation time (e.g., based on consistent cycle length and conduction velocity and/or based on neighboring electrograms, as described herein). Then, the detected cardiac activation that is closest in time to the most recent reference cardiac activation is automatically paired with the reference cardiac activation to compute LAT and create the LAT map.

By not using a RAI to compute the LAT, several advantages are realized when generating a LAT map. For example, the burden on a cardiac mapping system operator is reduced, as the operator does not have to select a RAI, adjust the RAI based on variable cardiac cycle lengths, or adjust the RAI when mapping during one-to-one atrial and ventricular cycle lengths. As will be evident from the following description, several other advantages are also realized.

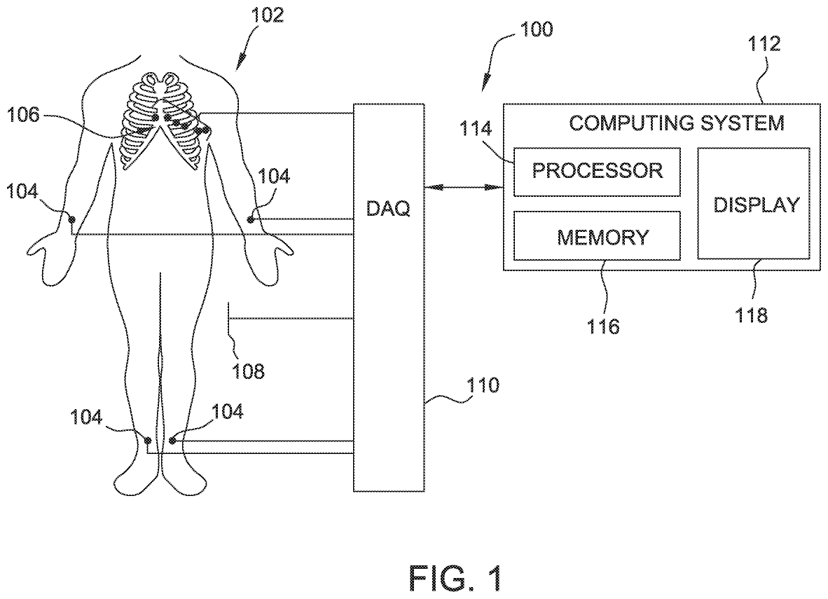

FIG. 1 is a schematic and block diagram of an ECG system 100 for conducting an ECG on a patient 102. ECG System 100 shown in FIG. 1 is sometimes referred to as a surface ECG that measures electrical activity of patient 102's heart using various cutaneous electrodes, including limb electrodes 104 and precordial electrodes 106. System 100, in certain embodiments may further include internal electrodes (not shown) inserted into patient 102 using a cardiac catheter. System 100 includes a common electrode 108 that, in certain embodiments, serves as a common reference for others of limb electrodes 104 and precordial electrodes 106, and, more specifically, any unipole electrodes among them.

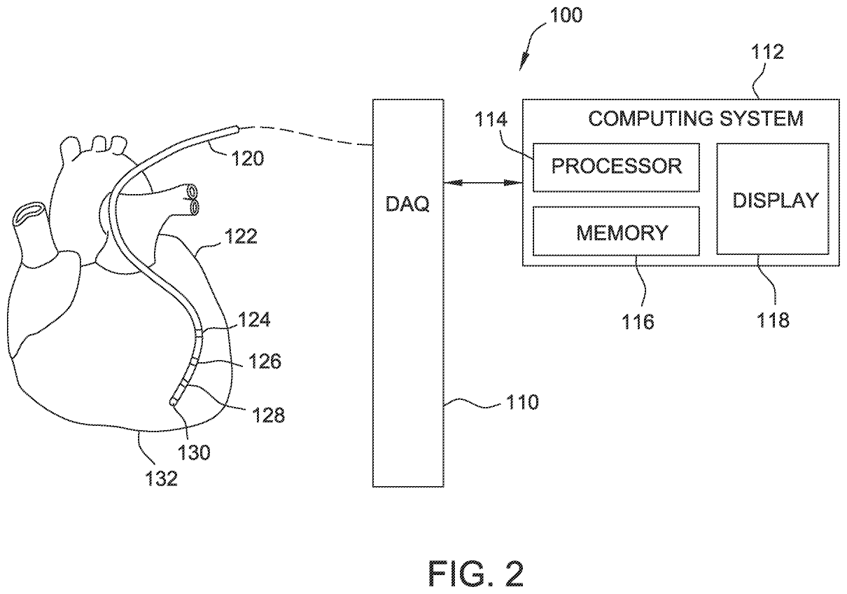

FIG. 2 is another schematic and block diagram of ECG system 100, including a catheter 120 having various catheter electrodes 124, 126, 128, and 130, sometimes referred to as distal electrodes. Catheter 120, in certain embodiments, may utilize a single catheter having numerous splines, each with multiple electrodes. In alternative embodiments, system 100 may utilize multiple catheters 120, each with multiple electrodes. In certain embodiments, catheter 120 is embodied in a high-density grid catheter, such as the EnSite™ Array™ non-contact mapping catheter of Abbott Laboratories. Catheter 120 is generally introduced to heart 122, vasculature, or ventricle 132 of patient 102 utilizing one or more introducers and using known procedures. Catheter 120 includes bipole electrodes and unipole electrodes.

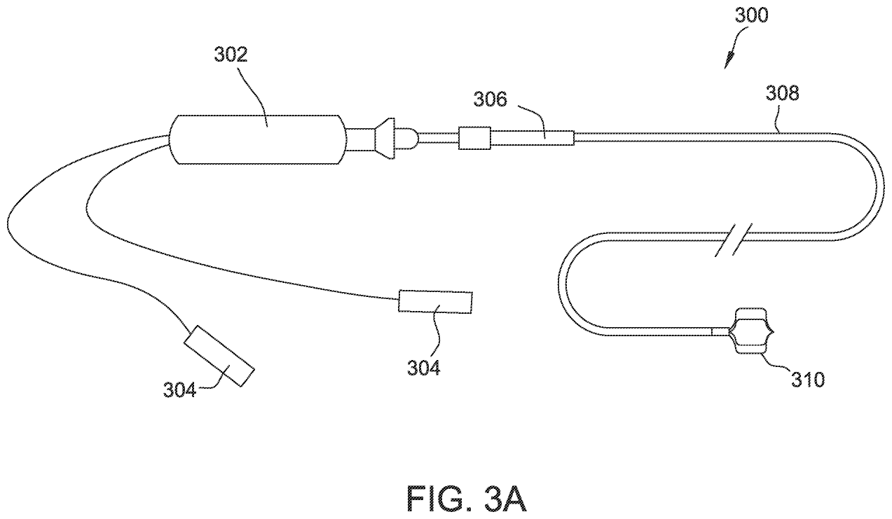

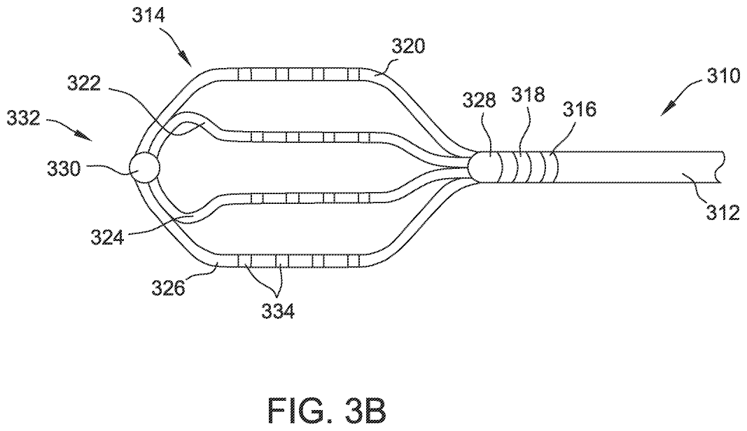

FIG. 3A is a schematic diagram of an exemplary catheter system 300. Catheter system 300 includes a handle 302 and connectors 304 disposed proximal to handle 302 for making electrical connections to an electronic mapping system or other suitable computing system. Catheter system 300 includes an introducer sheath 306 located distal to handle 302 that a surgeon may use to deliver a sheath 308 into the body of patient 102. Sheath 308 extends from introducer sheath 306. Catheter system 300 further includes an electrode assembly 310 that protrudes from the distal end of sheath 308. Catheter system 300 may be embodied, for example, and without limitation, in catheter systems described in U.S. Pat. No. 8,224,416, which is hereby incorporated by reference herein in its entirety.

FIG. 3B is a schematic diagram of an exemplary electrode assembly 310, for use in catheter system 300. Electrode assembly 310 includes a catheter body 312 coupled to a paddle 314. Catheter body 312 includes a first body electrode 316 and a second body electrode 318. Paddle 314 includes a first spline 320, a second spline 322, a third spline 324, and a fourth spline 326 coupled to catheter body 312 by a proximal coupler 328 and coupled to each other by a distal connector 330 at a distal end 332 of paddle 314. In one embodiment, first spline 320 and fourth spline 326 are one continuous segment and second spline 322 and third spline 324 are another continuous segment. In alternative embodiments, each of splines 320, 322, 324, and 326 are separate segments coupled to each other. Splines 320, 322, 324, and 326 include electrodes 334. Electrodes 334 may be embodied, for example, in ring electrodes evenly spaced along splines 320, 322, 324, and 326. In alternative embodiments, electrodes 334 may be embodied in point electrodes or any other suitable type of electrode.

Electrical activity produced by the heart manifests as small changes in charge of various cells of patient 102 that are detectable using specialized instrumentation, such as a data acquisition system (DAQ) 110 that is connected to surface ECG electrodes and the various electrodes of catheter 120. DAQ 110 includes various analog and digital circuits for sensing, conditioning, and relaying the electrogram signals generated at limb electrodes 104, precordial electrodes 106, and catheter electrodes to a computing system 112. Computing system 112 may be, for example, a cardiac mapping system.

Computing system 112 includes a processor 114, a memory 116, and a display 118. Computing system 112 may be embodied by the EnSite NavX™ system of Abbott Laboratories, which is capable of measuring electrical activity of patient 102's heart to generate electrical activity maps that are produced using the apparatus and methods described herein. Such electrical activity maps, in certain embodiments, may not be generated within computing system 112. Computing system 112 may further be embodied by other ECG systems, such as, for example, the CARTO® system of Biosense Webster, Inc., or the AURORA® system of Northern Digital Inc.

Computing system 112 is configured to receive multiple electrograms from DAQ 110 and present them on display 118 for viewing by a user, such as, for example, a physician, clinician, technician, or other user. Computing system 112 may further be configured to record the multiple electrograms in memory 116. Processor 114 is configured to process the multiple electrograms to determine an activation time for a given cardiac cycle. Such activation times are fundamental to producing electrical activity maps, such as the LAT map, the regular cycle length map, the voltage map, and the conduction velocity map.

As described herein, the systems and methods described herein enable generating LAT maps from electrograms without using a RAI. As will be appreciated by those of skill in the art, the electrograms used to generate LAT maps are acquired by at least one reference catheter that remains in a fixed position and at least one roving catheter that is moved between different locations and held at each location for a predetermined period of time.

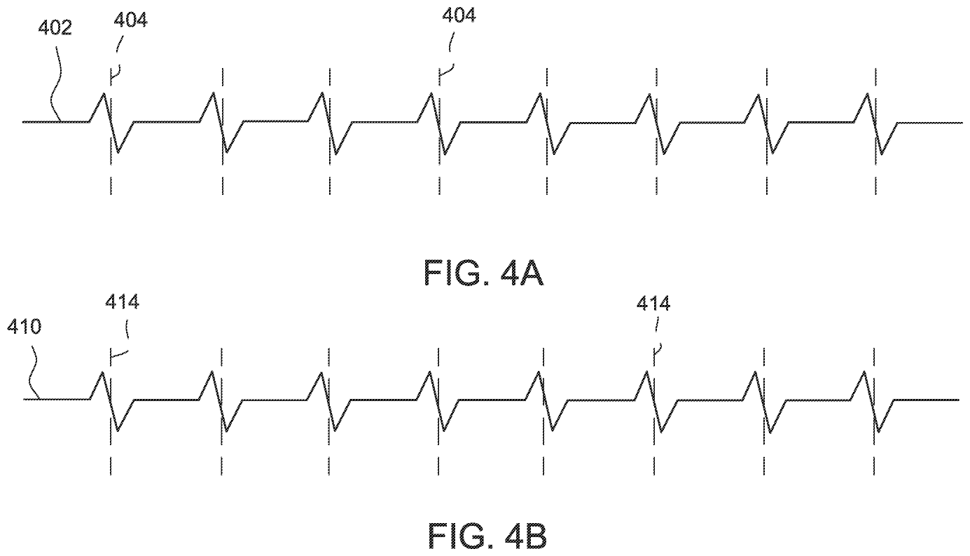

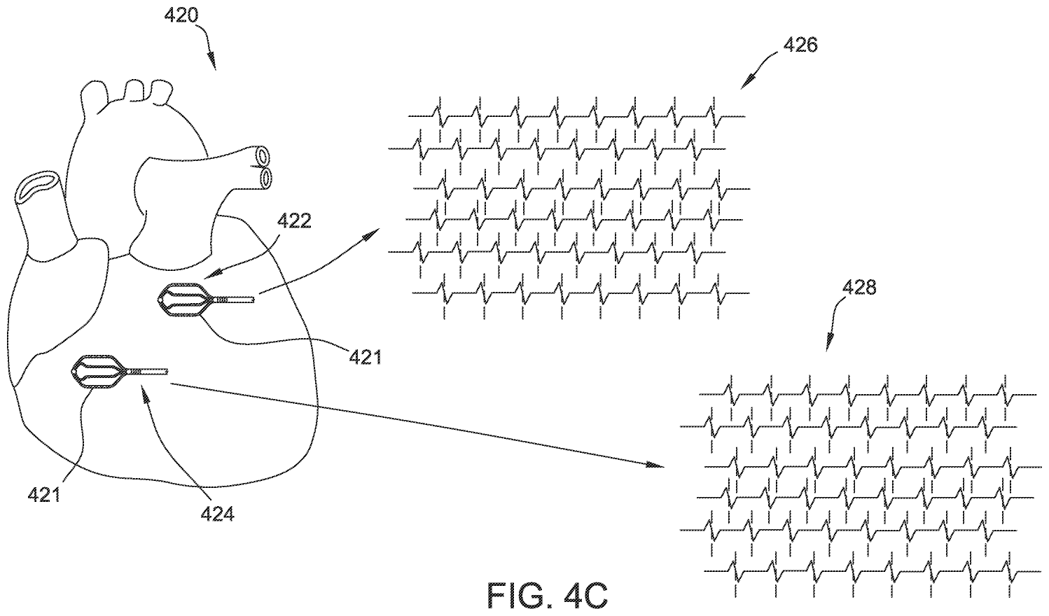

For example, FIG. 4A shows an electrogram 402 recorded by a reference catheter held stationary against cardiac tissue. A plurality of reference cardiac activations 404 are indicated on electrogram 402. As will be appreciated by those of skill in the art, reference cardiac activations 404 may be detected using any suitable technique. For example, reference cardiac activations 404 may be detected based on neighboring electrograms as discussed in International Application No. PCT/US2018/015224, filed Jan. 25, 2018, and/or based on consistent cycle length and conduction velocity as discussed in International Application No. PCT/US2018/015242, filed Jan. 25, 2018, each of which is incorporated by reference herein in its entirety.

FIG. 4B shows an electrogram 410 recorded by a roving catheter. Unlike the reference catheter, the roving catheter is moved between a plurality of different locations. At each location, the roving catheter is held against cardiac tissue for a user-specified time (e.g., in a range from 1 to 8 seconds). Electrogram 410 is an electrogram acquired by the roving catheter at one of the plurality of different locations. Similar to electrogram 402, a plurality of roving cardiac activations 414 are indicated on electrogram 410. As will be appreciated by those of skill in the art, roving cardiac activations 414 may be detected using any suitable technique. For example, roving cardiac activations 414 may be detected based on neighboring electrograms as discussed in International Application No. PCT/US2018/015224, filed Jan. 25, 2018, and/or based on consistent cycle length and conduction velocity as discussed in International Application No. PCT/US2018/015242, filed Jan. 25, 2018.

In practice, a roving catheter may be used to acquire electrograms at multiple locations, and a number of electrograms may be acquired by each roving catheter. For example, FIG. 4C is a schematic diagram 420 showing a roving catheter 421 positioned sequentially at a first position 422 and a second position 424 in a subject's heart. A plurality of first electrograms 426 are acquired using roving catheter 421 at first position 422, and a plurality of second electrograms 428 are acquired using roving catheter 421 at second position 424. Those of skill in the art will appreciate that cardiac activations on first and second electrograms 426 and 428 can be detected as described above.

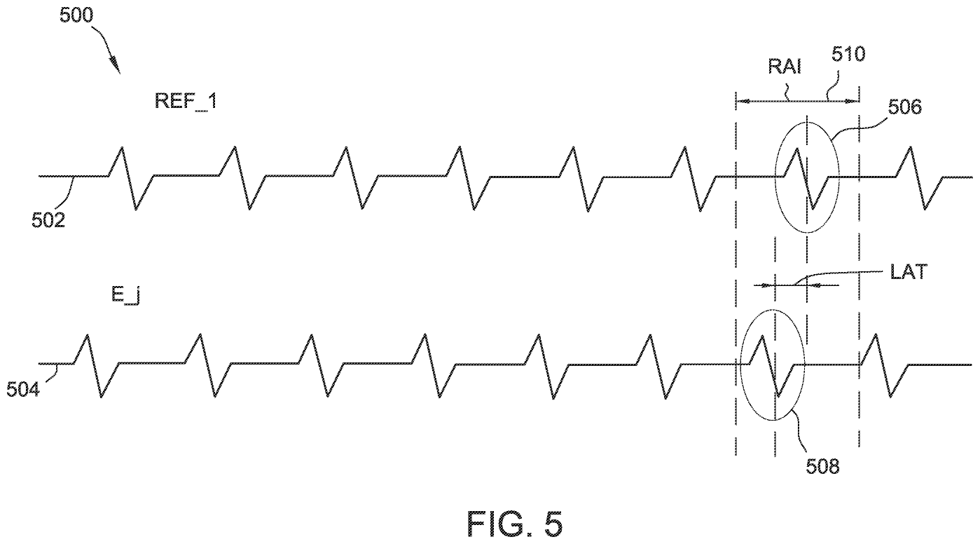

FIG. 5 is a diagram 500 illustrating computation of a LAT using a RAI (i.e., as done in at least some known existing cardiac mapping systems). Diagram 500 includes a reference electrogram 502 and a roving electrogram 504. Further, a reference cardiac activation 506 is indicated on reference electrogram 502, and a roving cardiac activation 508 is indicated on roving electrogram 504.

To compute a LAT, a RAI 510 (e.g., defined by a user) is placed relative to reference cardiac activation 506. As explained above, a cardiac cycle length (CCL) is measured and used to determine the time interval of RAI 510. However, there are multiple, debated approaches for determining the time interval, and there are multiple, debated approaches for placing RAI 510. Accordingly, both the time interval and placement of RAI 510 are somewhat subjective.

To compute the LAT, a mapping window is updated every time a cardiac activation (e.g., reference cardiac activation 506) is detected on reference electrogram 502, and the cardiac mapping system automatically identifies a roving cardiac activation that falls within RAI 510 (here, roving cardiac activation 508), and computes the LAT as the time interval between reference cardiac activation 506 and the roving cardiac activation. However, because RAI 510 may vary (due to different approaches in determining time interval and placement), the roving cardiac activation identified may vary, and the computed LAT may vary, depending on the placement and time interval of RAI 510. This results in different computed LATs (and thus different LAT maps) resulting from the same underlying electrograms, depending on the particular RAI used.

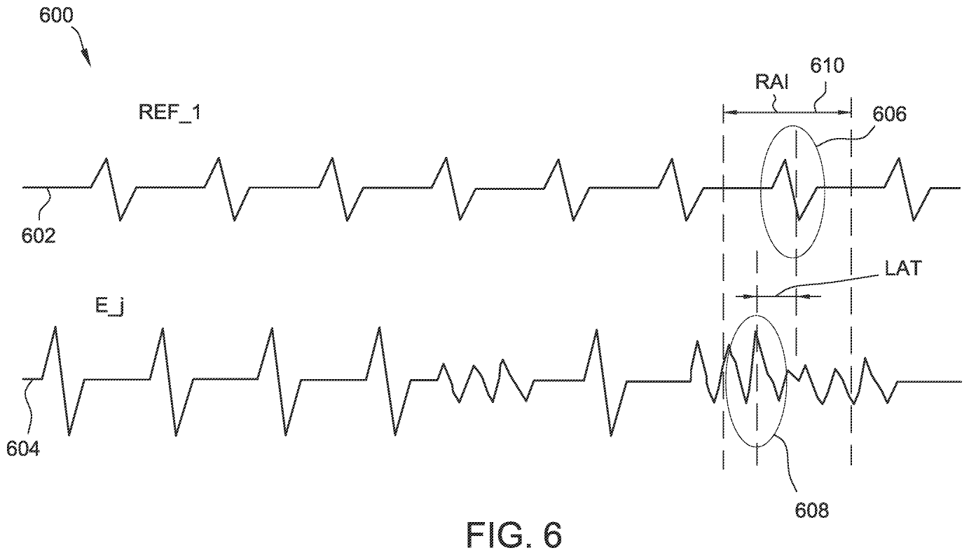

FIG. 6 is a diagram 600 illustrating another example of computation of a LAT using a RAI (i.e., as done in at least some known existing cardiac mapping systems). Diagram 600 includes a reference electrogram 602 and a roving electrogram 604. Further, a reference cardiac activation 606 is indicated on reference electrogram 602, and a roving cardiac activation 608 is indicated on roving electrogram 604. In this example, as shown in FIG. 6, roving electrogram 604 includes a number of inconsistent activations.

Similar to the example of FIG. 5, to compute a LAT, a RAI 610 is placed relative to reference cardiac activation 606. Then, the mapping window is updated every time a cardiac activation (e.g., reference cardiac activation 606) is detected on reference electrogram 602, and the cardiac mapping system automatically identifies a roving cardiac activation that falls within RAI 610, and computes the LAT as the time interval between reference cardiac activation 606 and the roving cardiac activation. Notably, in at least some known cardiac mapping systems, even though there are multiple inconsistent activations in roving electrogram 604, the system still attempts to automatically identify a corresponding roving cardiac activation (roving cardiac activation 608 in FIG. 6). The corresponding roving cardiac activation may be identified based on sharpness, peak value, and/or other criteria. However, this may result in identification of a false roving cardiac activation, instead of a roving cardiac activation that actually corresponds to reference cardiac activation 606 (resulting in an inaccurate computed LAT). Further, in at least some known systems, the LAT is still computed without notifying an operator that the computed LAT may be invalid due to the multiple inconsistent activations and chance that the actual corresponding roving cardiac activation was not selected.

Accordingly, as demonstrated by the examples shown in FIGS. 5 and 6, using a RAI may result in inaccurate LAT computations and inaccurate LAT maps generated therefrom.

As indicated above, the systems and methods described herein facilitate generating LAT maps without using a RAI. Generating LAT maps without using a RAI will now be described in detail.

In one embodiment, the user deploys at least one stationary reference catheter (REF) within the cardiac anatomy to record reference electrograms. In this discussion, each reference catheter is denoted REF_i, where i denotes the number of the particular reference catheter, and i=1, 2, . . . R, with R being the total number of reference catheters. As will be appreciated by those of skill in the art, each REF_i catheter measures one or more reference electrograms. Further, in this embodiment, REF_1 is used to compute LAT values.

The user also maneuvers a roving catheter (ROV) within the cardiac anatomy to measure electrograms from multiple anatomical sites (A). In this discussion, the roving electrograms are denoted E_i, wherein i denotes the number of the particular roving electrogram, and i=1, 2, . . . G, with G being the total number of roving electrograms. The roving catheter is held stationary at multiple anatomical sites. The anatomical sites used for mapping are denoted A_i, wherein i denotes the number of the particular anatomical site, and i=1, 2, . . . M, with M being the total number of anatomical sites used for mapping.

At each anatomical site A_i, the roving electrograms E_i are measured from the roving catheter over a user-defined segment length (SL). The SL may range, for example, from 0.1 seconds to 8 seconds. Alternatively, the SL may have any suitable length. Further, at each anatomical site A_i, the locations of the electrodes on the roving catheter (i.e., the electrodes used to record the electrograms) may be determined using any suitable techniques, as will be appreciated by those of skill in the art.

In one embodiment, the cardiac mapping system described herein (e.g., ECG system 100, computing system 112, etc.) operates in a mapping mode somewhat similar to the Non Cardiac Triggered (NCT) mode of the EnSite™ Velocity™ system of Abbott Laboratories. In this mapping mode, a map acquisition window is triggered independent of any cardiac activity and is triggered at an asynchronous interval. The asynchronous interval may be, for example within a range of approximately 100 milliseconds (ms) to 1000 ms. Alternatively, the asynchronous interval may be any suitable interval. In this discussion, the time of each trigger is denoted by T_i, where I=1, 2, . . . ∞.

At each T_i, a number of preprocessing steps occur. For these preprocessing steps, data is considered within an observation window having the SL. In one embodiment, the cardiac mapping system may set SL to be a time period of, for example, 5 seconds. In another embodiment, a user may set the length of SL. Further, the observation window may include an interval of time after T_i (e.g., 0.5 seconds) and an interval of time prior to T_i, such that the total length of the observation window is SL. Alternatively, the observation window and segment length may have any suitable length and placement, and may be set or defined using any suitable techniques.

As part of the preprocessing steps, within the observation window, dominant cycle length (DCLs) are computed, and cardiac activations are detected in the electrograms recorded by the REF_i and ROV catheters. In one embodiment, DCL is computed as described in International Application No. PCT/US2018/015224, filed Jan. 25, 2018, and/or International Application No. PCT/US2018/015242, filed Jan. 25, 2018.

Specifically, in one embodiment, the DCL is computed for the electrograms from the REF_i catheter. This will define the cycle length (CL) for each REF_i as DCL_REF_i, and also enables detecting reference cardiac activations in the electrograms recorded using the REF_i catheter. Further, a relative time between electrograms within REF_i may be computed. Specifically, for each DCL detection in REF_i, the time to the closest DCL detection in REF_j is computed (denoted herein as Δ_Time_REFi_REFj).

The DCL for each roving electrogram E_i is also calculated (denoted herein as DCL_E_i). The DCL_E_i values may be computed as described in International Application No. PCT/US2018/015224, filed Jan. 25, 2018, and/or International Application No. PCT/US2018/015242, filed Jan. 25, 2018. In a modified version, the reference cycle length (DCL_REF_i) is used to attempt to find a cluster of cycles within each roving electrogram E_i that most closely resembles the reference cycle length, if such a cluster exists. In some situations, it is possible that no matches will be found (e.g., if the reference electrograms and roving electrograms do not have similar DCLs). In some embodiments, the user can override the reference DCL value, DCL_REF_i. The computed DCL_E_i values define the cycle length for each E_i. This enables detecting cardiac activations in the roving electrograms which match the reference DCL (DCL_REF_i) most closely.

Accordingly, after the preprocessing steps have been performed, reference and roving cardiac activations have been detected in each of the associated reference and roving electrograms. At this point, at each T_i, the LAT is computable for each E_i.

Specifically, in one embodiment, the latest (i.e., most recent) reference cardiac activation in the REF_1 catheter is identified (denoted as REF_1_AT). To update display of the map acquisition window, it is useful to define the latest reference cardiac activation as the most recent reference cardiac activation that occurs at least a predetermined period of time (e.g., 0.5 seconds) before the current end of the electrogram. For example, it is useful to process data that is more recent than REF_1_AT in case there is a closer roving cardiac activation after REF_1_AT instead of before REF_1_AT.

In this embodiment, the roving cardiac activation closest in time to REF_1_AT is identified (denoted as E_j_AT). Then, the LAT for E_j (denoted as LAT_E_j) is computed as the different between REF_1_AT and E_j_AT (i.e., LAT_E_j=(REF_1_AT−E_j_AT)).

In some situations, a valid LAT may not be computable, and, accordingly, an ‘invalid LAT’ flat should be assigned to the corresponding roving electrogram E_i. For example, an invalid LAT flag may be assigned when i) REF_1_AT is not relatively recent (e.g., within 1 second of T_i), ii) there is no DCL value (i.e., no DCL_E_j value was found for electrogram E_j), or iii) LAT_E_j is greater than the cardiac cycle length.

In one embodiment, reference and roving electrograms are displayed to the user on a display device (e.g., display 118 shown in FIGS. 1 and 2). Further, the reference and roving activation times used to compute the LAT may also be displayed. For example, REF_1_AT may be indicated by a color-coded (e.g., yellow) detection marker, and E_j_AT may be indicated by another color-coded (e.g., green) detection marker. Further, a horizontal line representing LAT may be automatically drawn between the REF_1_AT detection marker and the E_j_AT detection marker. In some embodiments, the user can override the location of REF_1_AT and/or E_j_AT (e.g., by modifying the length of the horizontal line), resulting in the system computing an updated, user-defined LAT. In additional invalid LAT values may also be color-coded and clearly identified on the display device.

In one embodiment, the system is capable of aligning, different roving electrograms E_i and E_j, as well as other data acquired at different trigger times T_i and T_j. To align the data, the two corresponding reference activation times (REF_i_AT and REF_j_AT) are aligned. Then, other data collected at these two trigger times can then be analyzed as being collected simultaneously.

To generate LAT maps from computed LATs, a plurality of map points are collected. In one embodiment, the cardiac mapping system automatically collects an initial point if DCL_REF_1 and DCL_E_i are regular. In another embodiment, the user manually collects the initial map point (e.g., by operating computing system 112 (shown in FIGS. 1 and 2)). Subsequent map points are then collected with each trigger, T_i, for example, if the following criteria are met: i) DCL_REF_i is below a first user-specified threshold (e.g., within a range of approximately 0 ms to 50 ms, such as 20 ms), and ii) Δ_Time_REFi_REFj from the first map point is below a second user-specified threshold (e.g., within a range of approximately 0 ms to 50 ms, such as 20 ms). Those of skill in the art will appreciate that other suitable criteria may be used additionally or alternatively.

Using the collected map points, a LAT map is generated and displayed (e.g., on display 118 (shown in FIGS. 1 and 2). Specifically, the LAT map is generated and displayed based on the determined locations of electrodes on the roving catheters (as generally used in tracking systems), the computed LAT values, and color interpolation of those LAT values on geometry models. Notably, the systems and methods described herein may be used to compute LAT values for electrograms from all anatomical sites in a previously collected anatomical model, or from all anatomical sites while building a new anatomical model.

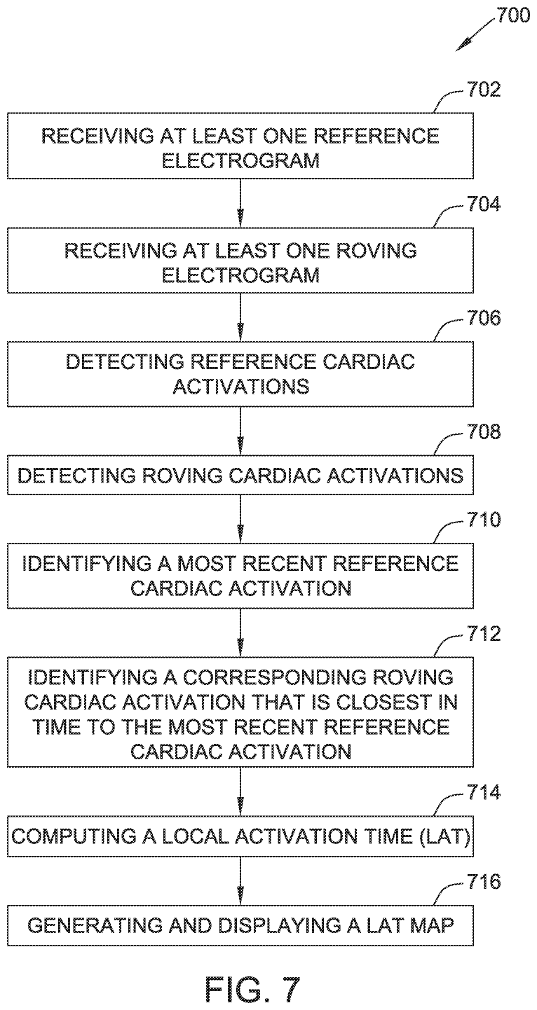

FIG. 7 is a flow diagram of an exemplary method 700 of generating LAT maps. Method 700 may be implemented, for example, using computing system 112 (shown in FIGS. 1 and 2). Method 700 includes receiving 702 at least one reference electrogram, and receiving 704 at least one roving electrogram. The at least one reference electrogram is recorded using at least one stationary reference catheter. The at least one roving electrogram is recorded using at least one roving catheter.

Method 700 further includes detecting 706 reference cardiac activations in the at least one reference electrogram, and detecting 708 roving cardiac activations in the at least one roving electrogram. The reference and roving cardiac activations may be detected 706, 708 using any suitable methods, such as, for example, those disclosed in International Application No. PCT/US2018/015224, filed Jan. 25, 2018, and/or International Application No. PCT/US2018/015242, filed Jan. 25, 2018.

In addition, method 700 includes identifying 710, at a trigger time, a most recent reference cardiac activation of the detected reference cardiac activations. Further, method 700 includes identifying 712 a corresponding roving cardiac activation of the detected roving cardiac activations that is closest in time to the most recent reference cardiac activation. Notably, in the systems and methods described herein, the corresponding roving cardiac activation is identified 712 independent of any RAI.

Method 700 further includes computing 714 a local activation time (LAT) as a time difference between the most recent reference cardiac activation and the corresponding roving cardiac activation. In addition, method 700 includes generating and displaying 716 a LAT map based on the computed LAT.

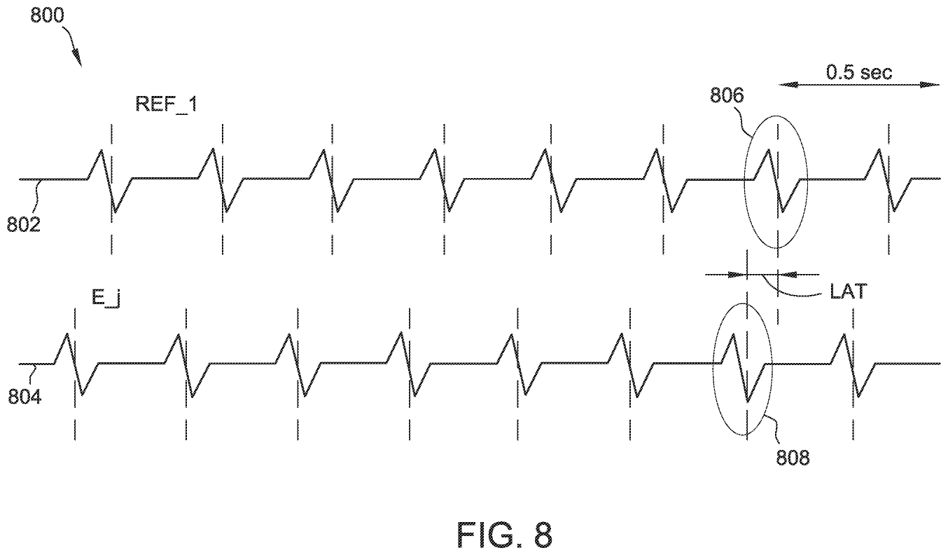

FIGS. 8-12 are diagrams illustrating operation of the systems and methods described herein. More specifically, FIG. 8 is a diagram 800 illustrating computation of a LAT without using a RAI. Diagram 800 includes a reference electrogram 802 and a roving electrogram 804. Further, a reference cardiac activation 806 is indicated on reference electrogram 802, and a roving cardiac activation 808 is indicated on roving electrogram 804. Using the systems and method described herein, reference cardiac activation 806 is identified as the most recent reference cardiac activation (here the most recent reference cardiac activation that occurs at least 0.5 seconds before the current end of reference electrogram 802).

Further, roving cardiac activation 808 is identified as being the closest in time to reference cardiac activation 806, and is thus identified as the corresponding roving cardiac activation. Accordingly, a LAT is computed as the time interval between reference cardiac activation 806 and roving cardiac activation 808.

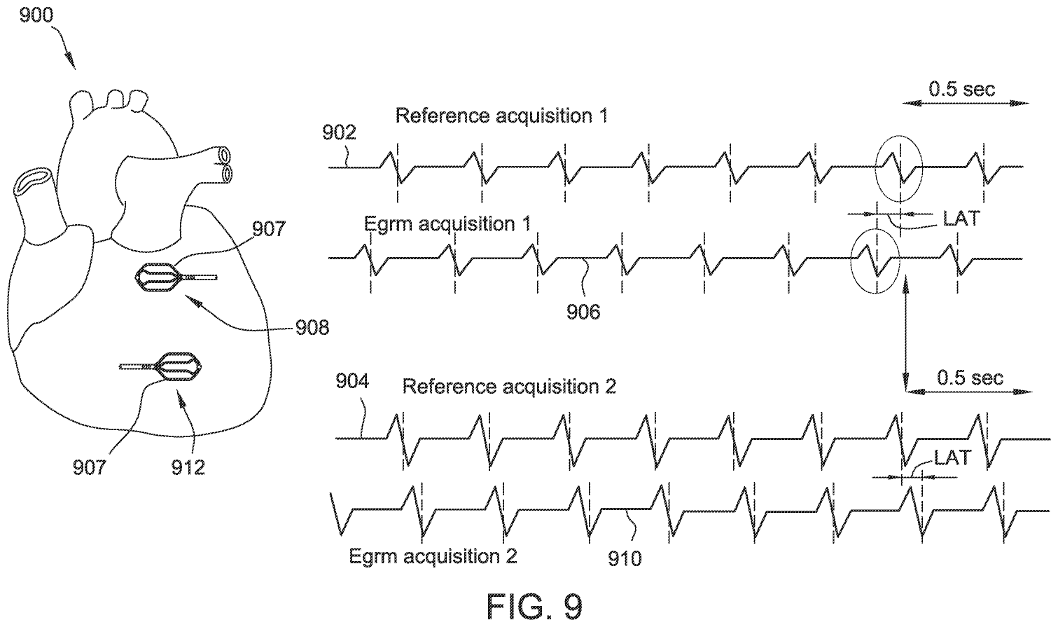

FIG. 9 is a diagram 900 illustrating computing LAT sequentially without using a RAI. Diagram 900 includes a first reference electrogram 902 and a second reference electrogram 904. Further, diagram 900 includes a first roving electrogram 906 captured using a roving catheter 907 at a first position 908 and a second roving electrogram 910 captured using roving catheter 907 positioned sequentially at a second position 912.

Because the cardiac cycle length is similar for first and second reference electrograms 902 and 904, first and second reference electrograms 902 and 904 can be aligned temporarily, as shown by the vertical double arrow in FIG. 9. Then, LATs can be computed for first and second roving electrograms 906 and 910 (e.g., as described in FIG. 8), and the sequentially acquired LATs can be appropriately color-coded in a resulting LAT map.

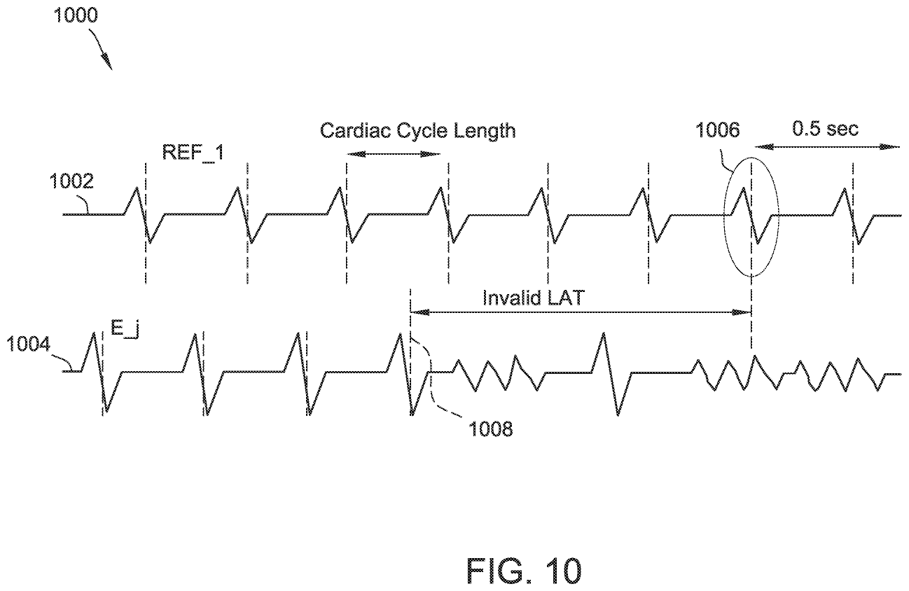

FIG. 10 is a diagram 1000 illustrating identifying an invalid LAT without using a RAI. Diagram 1000 includes a reference electrogram 1002 and a roving electrogram 1004. As shown in FIG. 10, roving electrogram 1004 includes a number of inconsistent deflections. Further, a reference cardiac activation 1006 is indicated on reference electrogram 1002, and a roving cardiac activation 1008 is indicated on roving electrogram 1004. Using the systems and method described herein, reference cardiac activation 1006 is identified as the most recent reference cardiac activation (here the most recent reference cardiac activation that occurs at least 0.5 seconds before the current end of reference electrogram 1002).

Further, roving cardiac activation 1008 is identified as being the closest in time to reference cardiac activation 1006, and is thus identified as the corresponding roving cardiac activation. Accordingly, a LAT is computed as the time interval between reference cardiac activation 1006 and roving cardiac activation 1008. Here, however, in contrast to the example of diagram 800 (shown in FIG. 8), the computed LAT is longer than the cardiac cycle length. Accordingly, the system flags the computed LAT as an invalid LAT and may update the display of the LAT accordingly.

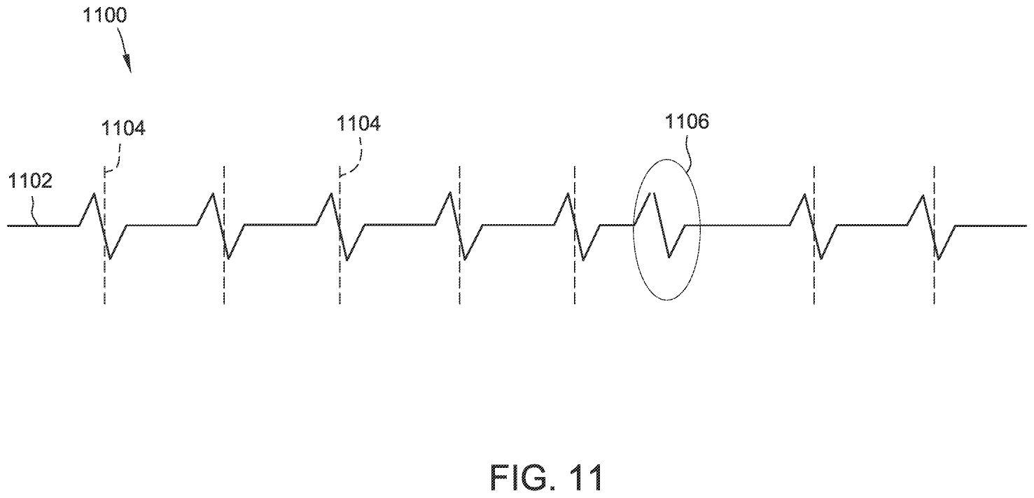

FIG. 11 is a diagram 1100 illustrating a reference electrogram 1102. A number of reference cardiac activations 1104 are detected for reference electrogram 1102. However, one deflection 1106 is not detected as a reference cardiac activation 1104. In this example, deflection 1106 does not share consistent characteristics (e.g., cycle length) with other deflections in reference electrogram 1102. Accordingly, deflection 1106 is not determined to include a reference cardiac activation 1104.

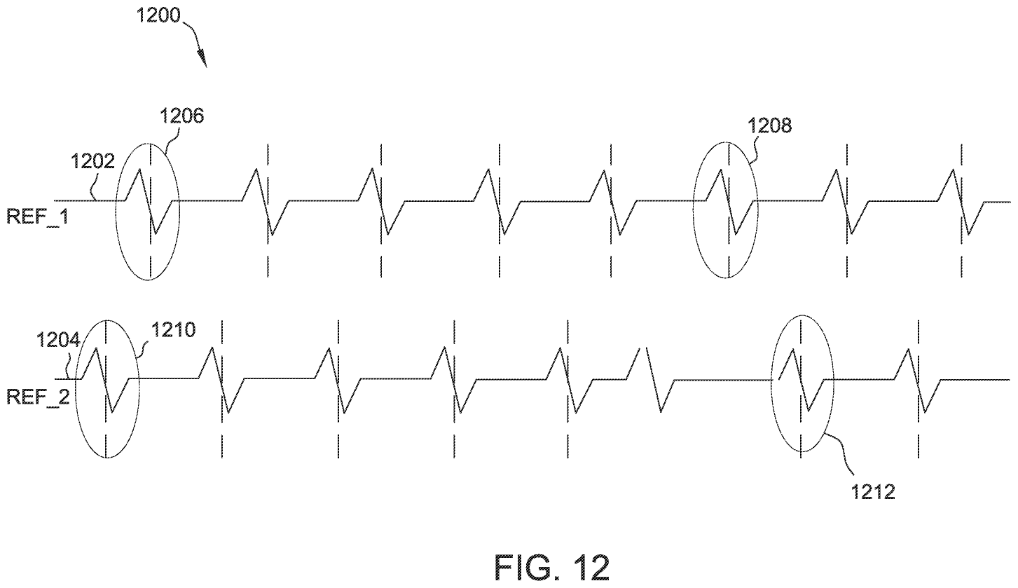

FIG. 12 is a diagram 1200 illustrating ignoring certain reference cardiac activations when computing LAT and generating an LAT map. Diagram 1200 includes a first reference electrogram 1202 and a second reference electrogram 1204. Further, a first reference cardiac activation 1206 and a second reference cardiac activation 1208 is indicated on first reference electrogram 1202, and a third reference cardiac activation 1210 and a fourth reference cardiac activation 1212 is indicated on second reference electrogram 1204. Intervals between reference cardiac activations on first reference electrogram 1202 and the closest reference cardiac activations on second reference electrogram 1204 are measured (e.g., the interval between first reference cardiac activation 1206 and third reference cardiac activation 1210). If a measured interval (e.g., the interval between second reference cardiac activation 1208 and fourth reference cardiac activation 1212) is dissimilar from previously measured intervals, the reference cardiac activations associated with that interval will be discarded from consideration when computing LAT and will not be used to generate the LAT map.

The embodiments described herein enable generating a local activation time (LAT) map. A method includes receiving at least one reference electrogram, receiving at least one roving electrogram, detecting cardiac activations in the at least one reference electrogram, and detecting roving cardiac activations in the at least one roving electrogram. The method further includes identifying, at a trigger time, a most recent reference cardiac activation of the detected reference cardiac activations, and identifying, a corresponding roving cardiac activation of the detected roving cardiac activations that is closest in time to the most recent reference cardiac activation. The corresponding roving cardiac activation is identified independent of any roving activation interval (RAI). The method further includes computing a LAT as a time difference between the most recent reference cardiac activation and the corresponding roving cardiac activation, and generating and displaying, a LAT map based on the computed LAT.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method of generating a local activation time (LAT) map, said method comprising:
receiving, at a computing system, at least one reference electrogram;
receiving, at the computing system, at least one roving electrogram;
detecting, using the computing system, reference cardiac activations in the at least one reference electrogram;
detecting, using the computing system, roving cardiac activations in the at least one roving electrogram;
identifying, using the computing system, at a trigger time, a most recent reference cardiac activation of the detected reference cardiac activations;
identifying, using the computing system, a corresponding roving cardiac activation of the detected roving cardiac activations that is closest in time to the most recent reference cardiac activation, the corresponding roving cardiac activation identified independent of any roving activation interval (RAI);
computing, using the computing system, a LAT as a time difference between the most recent reference cardiac activation and the corresponding roving cardiac activation; and
generating and displaying, using the computing system, a LAT map based on the computed LAT.

2. The method of claim 1, wherein identifying the most recent reference activation time comprises identifying the most recent reference activation time that occurs at least a predetermined period of time before the current end of the at least one reference electrogram.

3. The method of claim 1, further comprising:
comparing, using the computing system, the computed LAT to a cardiac cycle length; and
flagging, using the computing system, the computed LAT as an invalid LAT when the computed LAT is greater than the cardiac cycle length.

4. The method of claim 1, wherein detecting reference and roving cardiac activations comprises detecting reference and roving cardiac activations based on neighboring electrograms.

5. The method of claim 1, wherein detecting reference and roving cardiac activations comprises detecting reference and roving cardiac activations based on consistent cycle length and conduction velocity.

6. The method of claim 1, wherein the trigger time occurs repeatedly at an asynchronous interval.

7. The method of claim 1, wherein detecting reference and roving cardiac activations comprises detecting reference and roving cardiac activations within an observation window having a predefined segment length, the observation window including a first interval of time before the trigger time and a second interval of time after the trigger time.

8. A computing system for use in generating a local activation time (LAT) map, the computing system comprising:
a memory; and
a processor communicatively coupled to the memory, the processor configured to:
receive at least one reference electrogram;
receive at least one roving electrogram;
detect reference cardiac activations in the at least one reference electrogram;
detect roving cardiac activations in the at least one roving electrogram;
identify, at a trigger time, a most recent reference cardiac activation of the detected reference cardiac activations;
identify a corresponding roving cardiac activation of the detected roving cardiac activations that is closest in time to the most recent reference cardiac activation, the corresponding roving cardiac activation identified independent of any roving activation interval (RAI);
compute a LAT as a time difference between the most recent reference cardiac activation and the corresponding roving cardiac activation; and
generate and display a LAT map based on the computed LAT.

9. The computing system of claim 8, wherein to identify the most recent reference activation time, the processor is configured to identify the most recent reference activation time that occurs at least a predetermined period of time before the current end of the at least one reference electrogram.

10. The computing system of claim 8, wherein the processor is further configured to:
compare the computed LAT to a cardiac cycle length; and
flag the computed LAT as an invalid LAT when the computed LAT is greater than the cardiac cycle length.

11. The computing system of claim 8, wherein to detect reference and roving cardiac activations, the processor is configured to detect reference and roving cardiac activations based on neighboring electrograms.

12. The computing system of claim 8, wherein to detect reference and roving cardiac activations, the processor is configured to detect reference and roving cardiac activations based on consistent cycle length and conduction velocity.

13. The computing system of claim 8, wherein the trigger time occurs repeatedly at an asynchronous interval.

14. The computing system of claim 8, wherein to detect reference and roving cardiac activations, the processor is configured to detect reference and roving cardiac activations within an observation window having a predefined segment length, the observation window including a first interval of time before the trigger time and a second interval of time after the trigger time.

15. A cardiac mapping system for use in generating a local activation time (LAT) map, the system comprising:
at least one reference catheter;
at least one roving catheter; and
a computing system communicatively coupled to the at least one reference catheter and the at least one roving catheter, the computing system configured to:
receive at least one reference electrogram recorded by the at least one reference catheter;
receive at least one roving electrogram recorded by the at least one roving catheter;
detect reference cardiac activations in the at least one reference electrogram;
detect roving cardiac activations in the at least one roving electrogram;
identify, at a trigger time, a most recent reference cardiac activation of the detected reference cardiac activations;
identify a corresponding roving cardiac activation of the detected roving cardiac activations that is closest in time to the most recent reference cardiac activation, the corresponding roving cardiac activation identified independent of any roving activation interval (RAI);
compute a LAT as a time difference between the most recent reference cardiac activation and the corresponding roving cardiac activation; and
generate and display a LAT map based on the computed LAT.

16. The cardiac mapping system of claim 15, wherein to identify the most recent reference activation time, the computing system is configured to identify the most recent reference activation time that occurs at least a predetermined period of time before the current end of the at least one reference electrogram.

17. The cardiac mapping system of claim 15, wherein the computing system is further configured to:
compare the computed LAT to a cardiac cycle length; and
flag the computed LAT as an invalid LAT when the computed LAT is greater than the cardiac cycle length.

18. The cardiac mapping system of claim 15, wherein to detect reference and roving cardiac activations, the computing system is configured to detect reference and roving cardiac activations based on neighboring electrograms.

19. The cardiac mapping system of claim 15, wherein to detect reference and roving cardiac activations, the computing system is configured to detect reference and roving cardiac activations based on consistent cycle length and conduction velocity.

20. The cardiac mapping system of claim 15, wherein to detect reference and roving cardiac activations, the computing system is configured to detect reference and roving cardiac activations within an observation window having a predefined segment length, the observation window including a first interval of time before the trigger time and a second interval of time after the trigger time.
